(19) United States
(12) Patent Application Publication
(10) Pub. No.: US 2011/0218505 A1
(43) Pub. Date: Sep. 8, 2011

(54) ANTIEMETIC PATCH

(75) Inventors: Jui-Mei Lu, Hsinchu County (TW); Lih-Hua Lian, Hsinchu City (TW); Li-Fen Yao, Hsinchu City (TW)

(73) Assignees: GENOVATE BIOTECHNOLOGY CO., LTD., Hsin-Chu (TW); STANDARD CHEM & PHARM CO., LTD., Sinying City (TW); JIANBO INTERNATIONAL DEVELOPMENT LTD., Taipei (TW)

(21) Appl. No.: 13/128,522

(22) PCT Filed: Nov. 26, 2009
(86) PCT No.: PCT/CN2009/001323

§ 371 Date: May 10, 2011

Related U.S. Application Data
(60) Provisional application No. 61/118,728, filed on Dec. 1, 2008.

(51) Int. Cl. A61M 35/00

(52) U.S. Cl. 604/290; 604/304; 604/307

(57) ABSTRACT

An antiemetic patch is provided. The antiemetic patch includes a backing layer, a release liner, and a matrix layer disposed between the backing layer and the release liner. The matrix layer includes an antiemetic composition, and the antiemetic composition includes an antiemetic agent ranged, a permeation enhancer and an adhesive.

### REFERENCE TO RELATED APPLICATIONS

This application is a 35 U.S.C. §371 national phase application of PCT/CN2009/001323, which claims the benefit of U.S. provisional application No. 61/118,728 filed Dec. 1, 2008, both of which are incorporated by reference as if fully set forth.

### FIELD OF THE INVENTION

The present invention relates to an antiemetic patch, and more particularly to an antiemetic transdermal patch having a matrix type.

### BACKGROUND OF THE INVENTION

A transdermal patch achieves therapeutic effects by causing medical active ingredients to permeate into a user via the skin thereof in a certain rate, and the medical active ingredients would be carried through the whole body of the user due to the blood circulation. In addition to avoiding a first pass effect of a liver and increasing bioavailability of a medicine, the transdermal patch can overcome the inconvenience and the pain of intravenous injection, oral or rectal administrations. Since the medicine is administrated into a human body in a stable rate, a plasma concentration of the medicine in a therapeutically effective range could be sustained for a long period, and a stable therapeutic effect would be obtained. During a period of treatment by using the transdermal patch, if the patient feels uncomfortable, the treatment can be stopped immediately without the problems resulted from drug retention, and the convenience thereof may enhance the treatment compliance of the patient, avoid harmful results of relapse when forgetting to take medicine, and prevent young patients from being afraid of injection. Therefore, such a medical instrument without a need of an injection or oral administration plays an important role in drug delivery systems.

The transdermal patch mainly has a reservoir type and a matrix type. The transdermal patch of reservoir type may comprise the medicine having higher concentration by adding large amounts of alcohol or other solvents thereinto, but would often result in a skin irritation. Although the transdermal patch of matrix type comprises relatively lower concentration of medicine, the effective contact area thereof is larger, and the skin irritation can be reduced since the additive amount of other solvents is smaller. Currently, the mainstream of the transdermal patch is the matrix type due to the smaller area thereof, which makes the patient feel more comfortable and reduces the irritation of the skin.

Nausea and vomiting are most common and annoying side-effects occurred in a patient undergoing anticancer treatment. The mentioned side-effects may even cause a dehydration or malnutrition, and seriously affect therapeutic effects. The vomiting caused by chemotherapy includes acute, delayed or anticipatory emesis. Current dosage form for treating nausea and vomiting caused by chemotherapy includes oral administration, intravenous injection and rectal suppository. Intravenous injection has to be performed in the hospital and to be observed by medical personnel, and the patient feels uncomfortable due to inflammation or burn of the portion injected. Oral drugs must be given to the patient in multiple doses, have individual differences in efficacy, and especially have limitations to the patient having dysphagia disease. Rectal suppository not only has individual differences in efficacy, but also is more inconvenient in use, and thus is much unacceptable by the patient. Therefore, the transdermal patch, which is sustainable in a therapeutically effective range for a long period, is provided as a better choice for the patient.

Chemotherapy would facilitate generation of serotonin from cells, and serotonin would stimulate a 5-HT₃ (5-hydroxytryptamine 3) receptor to induce the vomiting. Therefore, a 5-HT₃ receptor antagonist could achieve antiemetic effect by interrupting the serotonin. It has been known that Granisetron has strong effect and high selectivity to the 5-HT₃ receptor and thus can be adopted to prevent and treat nausea and vomiting caused by anticancer chemotherapy or radiation treatment. Currently, the antiemetic agent, e.g. KYTRIL® (Granisetron), is an oral administration agent or an intravenous injection agent. For treating nausea and vomiting caused by anticancer chemotherapy, the dosage of KYTRIL® oral agent (or liquid agent) is 2 mg (or 10 ml) once daily, or 1 mg (or 5 ml) twice daily, and the treated patient has a peak plasma concentration of the agent being 6 ng/ml.

In the current commercial market, an antiemetic transdermal patch “Sancuso” has been developed by an England company, ProStrakan. Sancuso was permitted in the United States on Sep. 12, 2008, which uses Granisetron as its effective ingredient having a dosage of 3.1 mg/24 hr. The actual product of Sancuso is a patch having an area of 52 cm² and Granisetron of 34.3 mg, and the therapeutic effect thereof can be sustained for 5 days after being applied. According to the patent (EP1589956B) related to Sancuso, the acrylic-based adhesive thereof includes a non-acid hydroxyl functional group so that a certain permeation amount could be maintained without using a permeation enhancer. Sancuso has a drawback that the area of the patch is too large, and the patient will feel very uncomfortable when being applied for a long period. Accordingly, there is a need of an improvement for Sancuso.

Furthermore, another antiemetic transdermal patch is developed by a USA company, Abeille, as AB-1001. The features of AB-1001 are described in U.S. 2006/0263421 (A1). AB-1001 is a transdermal patch having a matrix type and including permeation enhancer being 0.5% to 15% by weight of the skin-contacting layer, and a plasma concentration of the medicine in a therapeutically effective range is provided for period of time from an onset time to 12 hours or more after the patch is removed.

Accordingly, there is a need to develop a novel antiemetic patch, which is designed to make a user feel more comfortable and the plasma concentration of the medicine in the user sustainable for a longer period.

### SUMMARY OF THE INVENTION

An antiemetic patch is provided to treat any vomiting symptoms resulted from anticancer chemotherapy, and is applied to a skin of a patient to achieve a stable plasma concentration of the medicine in a therapeutically effective range sustained for at least 5 days. Further, the transdermal patch of a matrix type provided in the present invention has a simple manufacture process and a small area, and is applied to the patient once while effective therapeutical concentration up to 5 days is achieved, which overcomes the inconvenience caused by conventional administrations.

In accordance with one aspect of the present invention, an antiemetic patch is provided. The antiemetic patch includes a backing layer, a release liner, and a matrix layer disposed between the backing layer and the release liner. The matrix layer includes an antiemetic composition.

Preferably, the antiemetic patch has an area ranged from 10 cm² to 100 cm², and more preferably the antiemetic patch has an area ranged from 10 cm² to 60 cm².

Preferably, the antiemetic agent includes a 5-HT₃ receptor antagonist. Further, the antiemetic agent may include one selected from a group consisting of Granisetron, Ondansetro, Palonosetron, Tropisetron, Dolasetron and a combination thereof.

Preferably, the antiemetic composition includes an antiemetic agent, a permeation enhancer and an adhesive. The antiemetic agent is ranged from 1% to 15% by weight of the antiemetic composition, the permeation enhancer is ranged from 1% to 10% by weight of the antiemetic composition, and the remaining component of the antiemetic composition is the adhesive.

Preferably, the permeation enhancer includes one selected from a group consisting of C2-C20 aliphatic alcohol, C10-C20 aliphatic carboxylic acid, C2-C10 amide, C2-C10 lactam and a combination thereof. The C2-C20 aliphatic alcohol may include one selected from a group consisting of a propylene glycol, a benzyl alcohol, an oleyl alcohol and a combination thereof. The C10-C20 aliphatic carboxylic acid may include an oleic acid. The C2-C10 amide may be one of a dimethylacetamide and an N-methyl-2-pyrrolidone.

Preferably, the adhesive is an insoluble pressure-sensitive adhesive including an acrylic-based polymer. The adhesive may include one selected from a group consisting of DURO-TAK 87-2516 manufactured by DURO-TAK (DURO-TAK®87-2516), DURO-TAK 87-2287 manufactured by DURO-TAK (DURO-TAK®87-2287), GELVA-737 manufactured by GELVA (GELVA®737) and GELVA-788 manufactured by GELVA (GELVA®788).

In accordance with another aspect of the present invention, an antiemetic patch is provided. The antiemetic patch includes an antiemetic composition including an antiemetic agent ranged from 1% to 15% by weight of the antiemetic composition. Preferably, the antiemetic composition further includes a permeation enhancer ranged from 1% to 10% by weight of the antiemetic composition, and an adhesive.

Preferably, the antiemetic patch has a matrix type, which includes a backing layer, a release liner, and a matrix layer. The matrix layer is disposed between the backing layer and the release liner and includes the antiemetic composition.

Preferably, the antiemetic patch has an area ranged from 10 cm² to 100 cm², and more preferably the antiemetic patch has an area smaller than 52 cm².

In accordance with a further aspect of the present invention, a method of treating an emesis of an individual is provided. The method includes a step of applying the antiemetic patch provided in the present invention to the individual, wherein the antiemetic agent has a plasma concentration sustainable in a therapeutically effective range for at least 5 days after the antiemetic patch is applied to the individual.

Preferably, the emesis is one selected from a group consisting of an acute, a delayed, an anticipatory emesis and a combination thereof. Furthermore, the individual has a skin and a mucosa, and the antiemetic patch is applied to at least one of the skin and the mucosa.

Preferably, a plasma concentration of the antiemetic agent has a maximum value of 0.48 ng/ml/cm² after the antiemetic patch is applied to an individual. Moreover, the antiemetic agent has a plasma concentration sustainable in a therapeutically effective range for at least 5 days after the antiemetic patch is applied to an individual, and the plasma concentration of the antiemetic agent in the therapeutically effective range has a value larger than 0.48 ng/ml/cm², and the treated individual has a peak plasma concentration of the antiemetic agent being larger than 6 ng/ml.

The above objects and advantages of the present invention will become more readily apparent to those ordinarily skilled in the art after reviewing the following detailed descriptions and accompanying drawings, in which:

### BRIEF DESCRIPTION OF THE DRAWINGS

FIG. 1 is a diagram showing an embodiment of the antiemetic patch of the present invention.

FIG. 2 is a diagram showing a drug flux amount of the formulation no. Gra-190.

FIG. 3 is a diagram showing a drug cumulative amount of the formulation no. Gra-190.

### DETAILED DESCRIPTION OF THE PREFERRED EMBODIMENT

The present invention will now be described more specifically with reference to the following embodiments. It is to be noted that the following descriptions of embodiments of this invention are presented herein for the purposes of illustration and description only; it is not intended to be exhaustive or to be limited to the precise form disclosed.

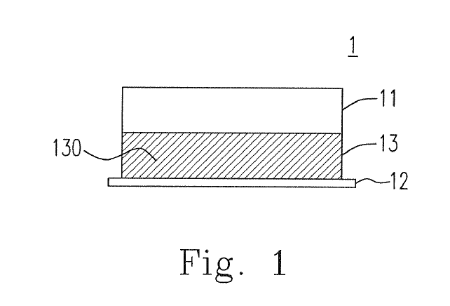

Please refer to FIG. 1, which is a diagram showing an embodiment of the antiemetic patch of the present invention. The antiemetic patch 1 includes a backing layer 11, a release liner 12 and a matrix layer 13 disposed between the backing layer 11 and the release liner 12, wherein the matrix layer 13 includes an antiemetic composition 130.

According to the mentioned embodiment, the antiemetic composition 130 includes an antiemetic agent and an adhesive, and may further include a permeation. In an example of the present invention, the antiemetic agent is ranged from 1% to 15% by weight of the antiemetic composition 130, the permeation enhancer is ranged from 1% to 10% by weight of the antiemetic composition 130, and the remaining component of the antiemetic composition 130 is the adhesive.

According to the mentioned embodiment, the antiemetic agent may include any pharmaceutical ingredient suitable for a transdermal administration, e.g. Granisetron, Ondansetro, Palonosetron, Tropisetron and Dolasetron. The adhesive may be an insoluble pressure-sensitive adhesive including an acrylic-based polymer. The adhesive may be one selected from a group consisting of DURO-TAK®87-2516, DURO-TAK®87-2287, GELVA®737 and GELVA®788. The permeation enhancer may be appropriately selected based on a pratical need. For example, the permeation enhancer may include one selected from a group consisting of C2-C20 aliphatic alcohol, C10-C20 aliphatic carboxylic acid, C2-C10 amide, C2-C10 lactam and a combination thereof. Specifically, the permeation enhancer may include one selected from a group consisting of a propylene glycol, a cineole, an oleic acid, an oleyl alcohol, a benzyl alcohol, a methyl laurate, a triacetin, a dimethylacetamide, a triethyl citrate, a dimethyl sulfoxide, a diethylene glycol monoethyl ether, an isopropyl myristate, an N-methyl-2-pyrrolidone and a combination thereof.

In the embodiments of the present invention, the antiemetic patch 1 is a transdermal patch having a matrix type, which has a single layer and a macromolecule matrix type. The antiemetic patch 1 is prepared by following steps. The permeation enhancer is well mixed with the antiemetic agent. The adhesive is added thereinto and these components are mixed until a clear adhesive liquid is provided. The clear adhesive liquid is applied on the release liner 12, which is then press-fitted with the backing layer 11 after a drying process, wherein the backing layer 11 is a protecting layer of a patch. Finally, the patch is divided into suotable sizes, e.g. 10 to 100 cm². Preferably, the final product of the antiemetic patch 1 has an area ranged from 10 cm² to 60 cm².

In the embodiments of the present invention, a test for drug flux from the transdermal patch by human skin, in vitro, is provided. A skin permeation device, including a Modifed Franz diffusion cell and one of a human cadaver skin and a membrane, is used to evaluate a drug flux amount. PBS (phosphate buffer saline) of pH 7.4 is used as an extract solution. The test procedure is described as follows.

A human cadaver skin is cut to a desired size (1.5 cm×1.5 cm) and mounted on a Modifed Franz diffusion cell. The release liner 12 is peeled away from the antiemetic patch 1 and placed on the human cadaver skin. A receptor solution is added to the diffusion cell and the assembly is maintained at 32 . The antiemetic patch 1 is sampled at each specific time point, and the drug flux concentration is analyzed by high performance liquid chromatograph (HPLC). Drug flux amont by skin, in vitro, per unit area of each hour is calculated (μg/cm²/hr).

A simulation research is performed as follows. It is hypothesized that the drug transmission from the patch to the skin is determined by Fick's Law:

$$\frac{\partial C}{\partial t} = D {∇}^{2} C ( \vec{r} , t ) \qquad Equation ( 1 )$$

In the Equation (1), C represents the drug concentration, and D represents the diffusion coefficient.

The drug concentration in the patch is C=Cₜ₌₀ when time is zero. Moreover, it is hypothesized that the drug is taken away by the blood upon diffusing into a border between the cornea layer and the dermis of the skin, and then is brought into the circle of whole body. In such a condition, the diffusion coefficient of the patch, Dp, and that of the cornea layer of the skin, Ds, can be obtained by solving the diffusion equation (i.e. Equation (1)). The mentioned diffusion coefficients are then applied to the diffusion mode of the patch when actually being used.

Furthermore, an equation for calculating the drug concentration in the plasma is provided as follows:

$$\frac{d C}{d t} = \frac{\partial C}{\partial x} {\vert}_{x = 0 , t = t} - {k}_{elim} C \qquad Equation ( 2 )$$

In the Equation

$$\frac{\partial C}{\partial x} {\vert}_{x = 0 , t = t}$$

represents a general amount of the transmitted drug, which is calculated by the diffusion equation, and kₑₗᵢₘ represents a metabolism coefficient of the drug. Accordingly, the change of the drug concentration in the plasma during different periods that the patch has been applied can be obtained.

Based on the data of the flux amount, per unit area in each hour, in the mentioned test, a maximun drug concentration in the plasma (ng/ml) in a human body can be evaluated by a computer simulation program, whereby predetermining an area of the patch which is able to stably provide a peak plasma concentration of the drug being 6 ng/ml for at least 5 days.

The following Examples A-1 to A-3 provide the results of the mentioned test (in vitro) for the drug flux from the antiemetic patch 1, including different ratios of the respective components in the antiemetic composition 130, by the human cadaver skin. Granisetron is used as the antiemetic agent of the antiemetic composition 130 in Examples A-1 to A-3, but it is not limited to this drug in a practical applictaion.

### EXAMPLE A-1

1. Please refer to Table A-1-1, which shows different formuations of the antiemetic patch of the present invention. As shown in Table A-1-1, the Granisetron base is ranged from 7% to 10% by weight of the antiemetic composition, the permeation enhancer is an oleic acid, and the adhesive is DURO-TAK®87-2516.

TABLE A-1-1

| | Composition (wt %) | | | | | |
|---|---|---|---|---|---|---|
| | Antiemetic | | | | | |
| | agent | Permeation enhancer | | | | Adhesive |
| | Granisetron | Oleyl | Oleic | Propylene | N-methyl-2- | DURO- |
| Formulation | base | alcohol | acid | glycol | pyrrolidone | TAK ®87-2516 |
| Gra-19 | 10 | — | 9 | — | — | 81 |
| Gra-57 | 10 | — | 11 | — | — | 79 |
| Gra-58 | 7 | — | 7 | — | — | 86 |
| Gra-59 | 7 | — | 9 | — | — | 84 |
| Gra-60 | 7 | — | 11 | — | — | 82 |
| Gra-61 | 8 | — | 7 | — | — | 85 |
| Gra-62 | 8 | — | 9 | — | — | 83 |
| Gra-63 | 8 | — | 11 | — | — | 81 |
| Gra-64 | 9 | — | 7 | — | — | 84 |
| Gra-65 | 9 | — | 9 | — | — | 82 |
| Gra-66 | 9 | — | 11 | — | — | 80 |

2. Please refer to Table A-1-2, which shows results of the test for the drug flux by the skin. The test results are presented by an average flux amount of the drug in each hour, and the sampling time points are 4 hr, 9 hr and 1-5 days, wherein a human cadaver skin No. 09580500107 is used in this test.

TABLE A-1-2

| | Drug | | | | | | | |
|---|---|---|---|---|---|---|---|---|
| | content | Flux (n = 6) μg/cm²/hr | | | | | | |
| Formulation | mg/30 cm² | 4 hr | 9 hr | Day 1 | Day 2 | Day 3 | Day 4 | Day 5 |
| Gra-19 | 0.816 | 0.86 | 8.60 | 12.9 | 11.72 | 8.88 | 7.45 | 5.67 |
| Gra-57 | 0.846 | 3.75 | 11.30 | 12.92 | 11.92 | 8.93 | 8.14 | 5.92 |
| Gra-58 | 0.581 | 1.65 | 9.13 | 11.32 | 10.07 | 6.64 | 5.56 | 3.67 |
| Gra-59 | 0.794 | 7.03 | 11.79 | 12.26 | 10.65 | 7.46 | 6.47 | 4.73 |
| Gra-60 | 0.620 | 0.79 | 5.12 | 8.33 | 9.00 | 6.90 | 6.65 | 5.07 |
| Gra-61 | 0.692 | 0.30 | 6.84 | 10.51 | 10.14 | 7.56 | 6.92 | 5.32 |
| Gra-62 | 0.950 | 0.21 | 5.77 | 10.77 | 10.55 | 7.92 | 7.02 | 4.87 |
| Gra-63 | 0.760 | 0 | 4.52 | 6.02 | 10.54 | 8.41 | 7.77 | 5.62 |
| Gra-64 | 0.978 | 0.30 | 6.94 | 7.06 | 11.60 | 8.95 | 8.06 | 6.18 |
| Gra-65 | 0.840 | 6.59 | 13.06 | 8.90 | 9.65 | 10.11 | 7.74 | 5.61 |
| Gra-66 | 1.004 | 0 | 5.74 | 12.38 | 12.87 | 10.25 | 9.49 | 7.20 |

3. Please refer to Table A-1-3, which shows the maximun drug concentration in the plasma (ng/ml) in a human body evaluated by the computer simulation program.

TABLE A-1-3

| | Formulation | | | | | | | | | | |
|---|---|---|---|---|---|---|---|---|---|---|---|
| | Gra- | Gra- | Gra- | Gra- | Gra- | Gra- | Gra- | Gra- | Gra- | Gra- | Gra- |
| | 19 | 57 | 58 | 59 | 60 | 61 | 62 | 63 | 64 | 65 | 66 |
| Cmax | 0.62 | 0.40 | 0.44 | 0.48 | 0.39 | 0.42 | 0.48 | 0.45 | 0.51 | 0.49 | 0.56 |
| (ng/ml/cm²) | | | | | | | | | | | |
| Tmax (hr) | 40 | 40 | 24 | 28 | 28 | 28 | 28 | 28 | 28 | 28 | 28 |

In the Example A-1, for example, based on the data of the flux amount, per unit area in each hour, of the formulation no. Gra-62, a maximun drug concentration in the plasma in a human body being 0.48 ng/ml/cm² can be evaluated by the computer simulation program. If the patch using said formulation has an area of 12.5 cm², a peak plasma concentration of the drug provided stably by the patch is predetermined to be 6 ng/ml for 5 days.

### EXAMPLE A-2

1. Please refer to Table A-2-1, which shows different formuations of the antiemetic patch of the present invention. As shown in Table A-2-1, the Granisetron base is ranged from 5% to 9% by weight of the antiemetic composition, the permeation enhancer is an oleic acid or a mixture including an oleic acid and an oleyl alcohol, and the adhesive is DURO-TAK®87-2516.

TABLE A-2-1

| | Composition (wt %) | | | | | |
|---|---|---|---|---|---|---|
| | Antiemetic | | | | | |
| | agent | Permeation enhancer | | | | Adhesive |
| | Granisetron | Oleyl | Oleic | Propylene | N-methyl-2- | DURO- |
| Formulation | base | alcohol | acid | glycol | pyrrolidone | TAK ®87-2516 |
| Gra-66 | 9 | — | 11 | — | — | 80 |
| Gra-131 | 5 | 3.5 | 3.5 | — | — | 88 |
| Gra-132 | 5 | 4.5 | 4.5 | — | — | 86 |
| Gra-133 | 5 | 5.5 | 5.5 | — | — | 84 |
| Gra-134 | 6 | 3.5 | 3.5 | — | — | 87 |
| Gra-135 | 6 | 4.5 | 4.5 | — | — | 85 |
| Gra-136 | 6 | 5.5 | 5.5 | — | — | 83 |

2. Please refer to Table A-2-2, which shows results of the test for the drug flux by the skin. The test results are presented by an average flux amount of the drug in each hour, and the sampling time points are 4 hr, 9 hr and 1-5 days, wherein a human cadaver skin No. 0782700112 is used in the test.

TABLE A-2-2

| | Drug | | | | | | | |
|---|---|---|---|---|---|---|---|---|
| | content | Flux (n = 6) μg/cm²/hr | | | | | | |
| Formulation | mg/30 cm² | 4 hr | 9 hr | Day 1 | Day 2 | Day 3 | Day 4 | Day 5 |
| Gra-66 | 1.004 | 1.61 | 9.49 | 12.21 | 9.07 | 9.43 | 7.91 | 5.55 |
| Gra-131 | 0.436 | 0 | 2.26 | 8.45 | 9.13 | 6.1 | 4.37 | 2.76 |
| Gra-132 | 0.485 | 1.6 | 6.36 | 10.74 | 9.1 | 7.27 | 4.92 | 2.07 |
| Gra-133 | 0.451 | 0 | 4.08 | 13.76 | 11.85 | 7.61 | 5.29 | 3.08 |
| Gra-134 | 0.564 | 0.98 | 8.66 | 13.43 | 11.37 | 6.68 | 5.18 | 3.06 |
| Gra-135 | 0.614 | 1.38 | 7.63 | 16.54 | 10.23 | 6.07 | 6.08 | 4.06 |
| Gra-136 | 0.547 | 0.94 | 7.06 | 8.66 | 13.06 | 9.01 | 5.64 | 3.76 |

3. Please refer to Table A-2-3, which shows the maximun drug concentration in the plasma (ng/ml) in a human body evaluated by the computer simulation program.

TABLE A-2-3

| | Formulation | | | | | | |
|---|---|---|---|---|---|---|---|
| | Gra- | Gra- | Gra- | Gra- | Gra- | Gra- | Gra- |
| | 66 | 131 | 132 | 133 | 134 | 135 | 136 |
| Cmax | 0.56 | 0.51 | 0.56 | 0.69 | 0.66 | 0.70 | 0.71 |
| (ng/ml/cm²) | | | | | | | |
| Tmax (hr) | 28 | 20 | 20 | 16 | 20 | 20 | 16 |

### EXAMPLE A-3

1. Please refer to Table A-3-1, which shows different formuations of the antiemetic patch of the present invention. As shown in Table A-3-1, the Granisetron base is ranged from 5-6% by weight of the antiemetic composition, the permeation enhancer is one of an oleic acid and a propylene glycol, and the adhesive is DURO-TAK®87-2516.

TABLE A-3-1

| | Composition (wt %) | | | | | |
|---|---|---|---|---|---|---|
| | Antiemetic | | | | | |
| | agent | Permeation enhancer | | | | Adhesive |
| | Granisetron | Oleyl | Oleic | Propylene | N-methyl-2- | DURO- |
| Formulation | base | alcohol | acid | glycol | pyrrolidone | TAK ®87-2516 |
| Gra-137 | 5 | — | — | 7 | — | 88 |
| Gra-138 | 5 | — | — | 9 | — | 86 |
| Gra-139 | 5 | — | — | 11 | — | 84 |
| Gra-140 | 6 | — | — | 7 | — | 87 |
| Gra-141 | 6 | — | — | 9 | — | 85 |
| Gra-142 | 6 | — | — | 11 | — | 83 |
| Gra-161 | 5 | — | 7 | — | — | 88 |
| Gra-162 | 5 | — | 9 | — | — | 86 |
| Gra-163 | 5 | — | 11 | — | — | 84 |
| Gra-164 | 6 | — | 7 | — | — | 87 |
| Gra-165 | 6 | — | 9 | — | — | 85 |
| Gra-166 | 6 | — | 11 | — | — | 83 |

2. Please refer to Table A-3-2, which shows results of the test for the drug flux by the skin. The test results are presented by an average flux amount of the drug in each hour, and the sampling time points are 4 hr, 9 hr, and 1-5 days, wherein a human cadaver skin No. 0709200108 is used in the test.

TABLE A-3-2

| | Drug | | | | | | | |
|---|---|---|---|---|---|---|---|---|
| | content | Flux (n = 6) μg/cm²/hr | | | | | | |
| Formulation | mg/30 cm² | 4 hr | 9 hr | Day 1 | Day 2 | Day 3 | Day 4 | Day 5 |
| Gra-137 | 0.494 | 2.58 | 4.02 | 6.94 | 8.18 | 5.68 | 4.24 | 3.48 |
| Gra-138 | 0.541 | 2.04 | 1.25 | 4.27 | 6.75 | 4.31 | 4.42 | 4.11 |
| Gra-139 | 0.516 | 0.55 | 1.16 | 4.80 | 6.98 | 4.88 | 4.29 | 4.03 |
| Gra-140 | 0.535 | 0.58 | 1.09 | 5.93 | 7.3 | 5.76 | 4.89 | 4.55 |
| Gra-141 | 0.630 | 0.97 | 2.02 | 6.01 | 7.8 | 3.93 | 5.3 | 4.85 |
| Gra-142 | 0.582 | 1.18 | 2.31 | 6.01 | 7.2 | 3.89 | 5.05 | 4.98 |
| Gra-161 | 0.519 | 2.06 | 3.85 | 8.15 | 8.66 | 5.59 | 4.45 | 3.02 |
| Gra-162 | 0.413 | 0.65 | 1.05 | 3.33 | 5.51 | 4.4 | 4.07 | 3.87 |
| Gra-163 | 0.517 | 0.41 | 0.63 | 2.8 | 5.81 | 4.82 | 4.08 | 3.95 |
| Gra-164 | 0.514 | 4.89 | 11.8 | 11.96 | 9.29 | 5.85 | 4.24 | 3.95 |
| Gra-165 | 0.498 | 0.86 | 1.79 | 6.16 | 8.25 | 3.99 | 5.06 | 4.44 |
| Gra-166 | 0.555 | 0.66 | 1.4 | 5.45 | 7.86 | 5.5 | 5.56 | 5.05 |

3. Please refer to Table A-3-3, which shows the maximun drug concentration in the plasma (ng/ml) in a human body evaluated by the computer simulation program.

TABLE A-3-3

| Formulation | Gra137 | Gra138 | Gra139 | Gra140 | Gra141 | Gra142 | Gra161 | Gra162 | Gra163 | Gra164 | Gr [symbol] |
|---|---|---|---|---|---|---|---|---|---|---|---|
| Cmax | 0.20 | 0.20 | 0.17 | 0.19 | 0.19 | 0.19 | 0.21 | 0.15 | 0.16 | 0.24 | 0 [symbol] |
| (ng/ml/cm²) | | | | | | | | | | | |
| Tmax (hr) | 32 | 32 | 36 | 36 | 36 | 36 | 32 | 36 | 36 | 32 | 3 [symbol] |

[symbol]

indicates data missing or illegible when filed

The following Examples B-1 to B-3 provide results of the test (in vitro) for the drug flux from the antiemetic patch 1, including different ratios of the respective components in the antiemetic composition 130, by an artificial membrane (CoTran 9728 Membrane (3M, FEB08 B#010)). Granisetron is used as the antiemetic agent of the antiemetic composition 130 in Examples B-1 to B-3, but it is not limited to this drug in a practical applictaion.

### EXAMPLE B-1

1. Please refer to Table B-1-1, which shows different formuations of the antiemetic patch of the present invention. As shown in Table B-1-1, the Granisetron base is ranged from 5-7% by weight of the antiemetic composition, the permeation enhancer includes various combinations of an oleic acid, an oleyl alcohol and a propylene glycol, and the adhesive is DURO-TAK®087-2516.

TABLE B-1-1

| | Composition (wt %) | | | | | |
|---|---|---|---|---|---|---|
| | Antiemetic | | | | | |
| | agent | Permeation enhancer | | | | Adhesive |
| | Granisetron | Oleyl | Oleic | Propylene | N-methyl-2- | DURO- |
| Formulation | base | alcohol | acid | glycol | pyrrolidone | TAK ®87-2516 |
| Gra-59 | 7 | — | 9 | — | — | 84 |
| Gra-177 | 5 | 7 | — | — | — | 88 |
| Gra-177-01 | 5 | 6 | 1 | — | — | 88 |
| Gra-177-02 | 5 | 5 | 2 | — | — | 88 |
| Gra-177-12 | 5 | 3 | — | 4 | — | 88 |
| Gra-177-13 | 5 | 2 | — | 5 | — | 88 |
| Gra-177-14 | 5 | 1 | — | 6 | — | 88 |
| Gra-177-15 | 5 | 8 | 1 | — | — | 86 |
| Gra-177-16 | 5 | 7 | 2 | — | — | 86 |
| Gra-177-17 | 5 | 6 | 3 | — | — | 86 |
| Gra-177-33 | 5 | 10 | 1 | — | — | 84 |
| Gra-177-34 | 5 | 9 | 2 | — | — | 84 |
| Gra-177-73 | 5 | 4 | 1 | — | — | 90 |

2. Please refer to Table B-1-2, which shows results of the test for the drug flux by the membrane. The test results are presented by an average flux amount of the drug in each hour, and the sampling time points include 1, 2 and 3 days.

TABLE B-1-2

| | Drug content | | | |
|---|---|---|---|---|
| | (n = 3) | Flux (n = 6) μg/cm²/hr | | |
| Formulation | mg/30 cm² | Day 1 | Day 2 | Day 3 |
| Gra-59 | 19.06 | 3.28 | 2.57 | 2.32 |
| Gra-177 | 13.38 | 8.35 | 6.32 | 4.33 |
| Gra-177-01 | 11.22 | 6.16 | 4.37 | 3.21 |
| Gra-177-02 | 12.33 | 5.18 | 4.33 | 3.69 |
| Gra-177-12 | 12.21 | 6.31 | 4.65 | 3.37 |
| Gra-177-13 | 12.00 | 5.55 | 4.36 | 3.3 |
| Gra-177-14 | 11.52 | 5.79 | 4.56 | 3.3 |
| Gra-177-15 | 11.94 | 8.32 | 6.39 | 3.99 |
| Gra-177-16 | 12.33 | 5.69 | 4.58 | 3.73 |
| Gra-177-17 | 10.59 | 5.82 | 4.77 | 3.12 |
| Gra-177-33 | 13.44 | 8.86 | 6.70 | 4.37 |
| Gra-177-34 | 13.08 | 8.20 | 6.78 | 4.58 |
| Gra-177-73 | 12.97 | 8.34 | 6.57 | 4.48 |

### EXAMPLE B-2

1. Please refer to Table B-2-1, which shows different formuations of the antiemetic patch of the present invention. As shown in Table B-2-1, the Granisetron base is ranged from 4-7% by weight of the antiemetic composition, the permeation enhancer includes various combinations of an oleic acid, an oleyl alcohol and an N-methyl-2-pyrrolidone, and the adhesive is DURO-TAK®87-2516.

TABLE B-2-1

| | Composition (wt %) | | | | | |
|---|---|---|---|---|---|---|
| | Antiemetic | | | | | |
| | agent | Permeation enhancer | | | | Adhesive |
| | Granisetron | Oleyl | Oleic | Propylene | N-methyl-2- | DURO- |
| Formulation | base | alcohol | acid | glycol | pyrrolidone | TAK ®87-2516 |
| Gra-59 | 7 | — | 9 | — | — | 84 |
| Gra-177 | 5 | 7 | — | — | — | 88 |
| Gra-177-85 | 4 | 5 | 1 | — | — | 90 |
| Gra-177-87 | 4 | 7 | 1 | — | — | 88 |
| Gra-177-91 | 5 | — | — | — | 9 | 86 |
| Gra-177-92 | 4 | — | — | — | 5 | 91 |
| Gra-177-93 | 4 | — | — | — | 7 | 89 |
| Gra-177-94 | 4 | — | — | — | 9 | 87 |

2. Please refer to Table B-2-2, which shows results of the test for the drug flux by the membrane. The test results are presented by an average flux amount of the drug in each hour, and the sampling time points include 1, 2 and 3 days.

TABLE B-2-2

| | Drug content | | | |
|---|---|---|---|---|
| | (n = 3) | Flux (n = 6) μg/cm²/hr | | |
| Formulation | mg/30 cm² | Day 1 | Day 2 | Day 3 |
| Gra-59 | 19.06 | 2.82 | 2.30 | 1.91 |
| Gra-177 | 12.83 | 6.64 | 5.34 | 3.75 |
| Gra-177-85 | 8.87 | 4.95 | 4.07 | 2.95 |
| Gra-177-87 | 9.88 | 5.24 | 4.18 | 3.20 |
| Gra-177-91 | 12.11 | 5.90 | 4.60 | 3.00 |
| Gra-177-92 | 8.84 | 4.24 | 3.43 | 2.33 |
| Gra-177-93 | 8.78 | 4.02 | 3.19 | 2.16 |
| Gra-177-94 | 7.56 | 3.93 | 3.03 | 2.07 |

### EXAMPLE B-3

1. Please refer to Table B-3-1, which shows different formuations of the antiemetic patch of the present invention. As shown in Table B-3-1, the Granisetron base is ranged from 3-4% by weight of the antiemetic composition, the permeation enhancer is an oleyl alcohol, and the adhesive is DURO-TAK®87-2516.

TABLE B-3-1

| | Composition (wt %) | | | | | |
|---|---|---|---|---|---|---|
| | Antiemetic | | | | | |
| | agent | Permeation enhancer | | | | Adhesive |
| | Granisetron | Oleyl | Oleic | Propylene | N-methyl-2- | DURO- |
| Formulation | base | alcohol | acid | glycol | pyrrolidone | TAK ®87-2516 |
| Gra-190 | 4 | 5 | — | — | — | 91 |
| Gra-191 | 3 | 7 | — | — | — | 90 |

2. Please refer to Table B-3-2, which shows results of the test for the drug flux by the membrane of formulation no. Gra-190. The test results are presented by an average flux amount of the drug in each hour, and the sampling time points include 1, 2, 3, 4 and 5 days.

TABLE B-3-2

| | Formulation No. Gra-190 | | | | | |
|---|---|---|---|---|---|---|
| Sampling | | | | Cumulative | | |
| time | Flux | | CV | amount | | CV |
| (day) | (μg/cm²/hr) | Stdev | (%) | (μg/cm²) | Stdev | (%) |
| 1 | 3.25 | 0.23 | 7.0 | 77.79 | 5.48 | 7.0 |
| 2 | 2.86 | 0.15 | 5.2 | 146.53 | 8.35 | 5.7 |
| 3 | 2.60 | 0.19 | 7.3 | 208.82 | 12.14 | 5.8 |
| 4 | 2.16 | 0.13 | 6.0 | 260.65 | 14.93 | 5.7 |
| 5 | 1.75 | 0.12 | 6.7 | 302.73 | 17.08 | 5.7 |

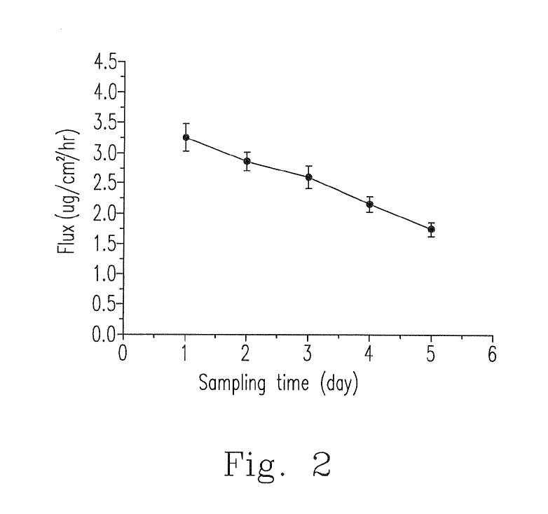

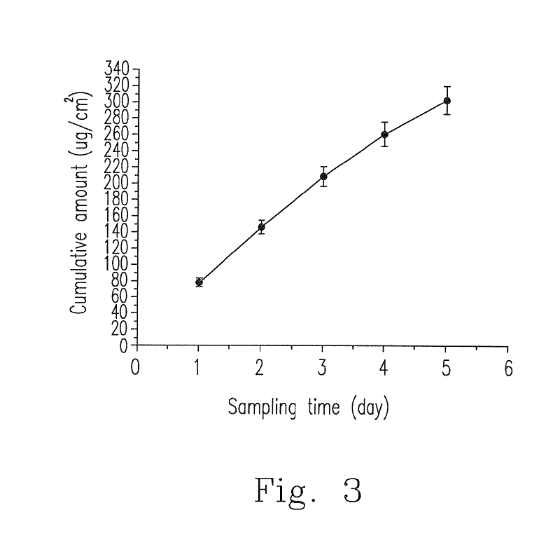

Please refer to FIG. 2, which is a diagram showing a drug flux amount of the formulation no. Gra-190. Furthermore, FIG. 3 is a diagram showing a drug cumulative amount of the formulation no. Gra-190.

The following Examples C-1 to C-6 provide results of the test (in vivo) for the drug flux from the antiemetic patch 1, having different formulations of the antiemetic composition 130 provided in Examples A-1 to B-3, by healthy human subjects.

### EXAMPLE C-1

There are three healthy human subjects in this test. The formulation no. Gra-59, which includes Granisetron (7%), an oleic acid (9%) and the adhesive DURO-TAK®87-2516 (84%), is applied. The size of the patch is designed to be 30 cm², and the test period is 48 hours.

Please refer to Table C-1, which shows the test results of the present example.

TABLE C-1

| Sampling Time | Drug Concentration in |
|---|---|
| (hr) | Plasma (ng/ml) |
| 0 | 0 |
| 24 | 2.00 |
| 48 | 4.26 |

### EXAMPLE C-2

There are three healthy human subjects in this test. The formulation no. Gra-177, which includes Granisetron (5%), an oleyl alcohol (7%) and the adhesive DURO-TAK®87-2516 (88%), is applied. The size of the patch is designed to be 30 cm², and the test period is 48 hours.

Please refer to Table C-2, which shows the test results of the present example.

TABLE C-2

| Sampling Time | Drug Concentration in |
|---|---|
| (hr) | Plasma (ng/ml) |
| 0 | 0 |
| 24 | 7.52 |
| 48 | 5.87 |

### EXAMPLE C-3

There are two healthy human subjects in this test. The formulation no. Gra-191, which includes Granisetron (3%), an oleyl alcohol (7%) and the adhesive DURO-TAK®87-2516 (90%), is applied. The size of the patch is designed to be 30 cm², and the test period is 48 hours.

Please refer to Table C-3, which shows the test results of the present example.

TABLE C-3

| Sampling Time | Drug Concentration in |
|---|---|
| (hr) | Plasma (ng/ml) |
| 0 | 0 |
| 24 | 0.69 |
| 48 | 1.25 |

### EXAMPLE C-4

There are three healthy human subjects in this test. The formulation no. Gra-190, which includes Granisetron (4%), an oleyl alcohol (5%) and the adhesive DURO-TAK®87-2516 (91%), is applied. The size of the patch is designed to be 30 cm², and the test period is 52 hours.

Please refer to Table C-4, which shows the test results of the present example.

TABLE C-4

| Sampling Time | Drug Concentration in |
|---|---|
| (hr) | Plasma (ng/ml) |
| 0 | 0 |
| 28 | 2.51 |
| 52 | 3.70 |

### EXAMPLE C-5

There are three healthy human subjects in this test. The formulation no. Gra-190, which includes Granisetron (4%), an oleyl alcohol (5%) and the adhesive DURO-TAK®87-2516 (91%), is applied. The size of the patch is designed to be 60 cm², and the test period is 72 hours.

Please refer to Table C-5, which shows the test results of the present example.

TABLE C-5

| Sampling Time | Drug Concentration in |
|---|---|
| (hr) | Plasma (ng/ml) |
| 0 | 0 |
| 24 | 5.37 |
| 48 | 6.91 |
| 72 | 4.60 |

### EXAMPLE C-6

There are seven healthy human subjects in this test. The formulation no. Gra-190, which includes Granisetron (4%), an oleyl alcohol (5%) and the adhesive DURO-TAK®87-2516 (91%), is applied. The size of the patch is designed to be 60 cm², and the test period is 156 hours.

Please refer to Table C-6, which shows the test results of the present example.

TABLE C-6

| Sampling Time | Drug Concentration in |
|---|---|
| (hr) | Plasma (ng/ml) |
| 0 | 0 |
| 12 | 0.60 |
| 24 | 1.69 |
| 36 | 2.22 |
| 48 | 1.84 |
| 60 | 1.55 |
| 72 | 1.25 |
| 96 | 1.09 |
| 120 | 1.21 |
| 126 | 0.86 |
| 132 | 0.74 |
| 156 | 0.39 |

While the invention has been described in terms of what is presently considered to be the most practical and preferred embodiments, it is to be understood that the invention needs not be limited to the disclosed embodiments. On the contrary, it is intended to cover various modifications and similar arrangements included within the spirit and scope of the appended claims which are to be accorded with the broadest interpretation so as to encompass all such modifications and similar structures.

What is claimed is:

1. An antiemetic patch, comprising:
a backing layer;
a release liner; and
a matrix layer disposed between the backing layer and the release liner, and including an antiemetic composition having an antiemetic agent ranged from 1% to 15% by weight of the antiemetic composition.

2. The antiemetic patch as claimed in claim 1, having an area ranged from 10 cm² to 100 cm².

3. The antiemetic patch as claimed in claim 1, having an area ranged from 10 cm² to 60 cm².

4. The antiemetic patch as claimed in claim 1, wherein the antiemetic agent includes a 5-HT₃ receptor antagonist.

5. The antiemetic patch as claimed in claim 1, wherein the antiemetic agent includes one selected from a group consisting of Granisetron, Ondansetro, Palonosetron, Tropisetron, Dolasetron and a combination thereof.

6. The antiemetic patch as claimed in claim 1, wherein the antiemetic composition further has a permeation enhancer and an adhesive.

7. The antiemetic patch as claimed in claim 6, wherein the permeation enhancer is ranged from 1% to 10% by weight of the antiemetic composition.

8. The antiemetic patch as claimed in claim 6, wherein the permeation enhancer includes one selected from a group consisting of C2-C20 aliphatic alcohol, C10-C20 aliphatic carboxylic acid, C2-C10 amide, C2-C10 lactam and a combination thereof.

9. The antiemetic patch as claimed in claim 8, wherein:
the C2-C20 aliphatic alcohol includes one selected from a group consisting of a propylene glycol, a benzyl alcohol, an oleyl alcohol and a combination thereof;
the C10-C20 aliphatic carboxylic acid includes an oleic acid; and
the C2-C10 amide is one of dimethvlacetamide and an N-methyl-2-pyrrolidone.

10. The antiemetic patch as claimed in claim 6, wherein the adhesive is an insoluble pressure-sensitive adhesive including acrylic-based polymer.

11. The antiemetic patch as claimed in claim 6, wherein the adhesive includes one selected from a group consisting of DURO-TAK 87-2516 manufactured by DURO-TAK, DURO-TAK 87-2287 manufactured by DURO-TAK, GELVA-737 manufactured by GELVA and GELVA-788 manufactured by GELVA.

12. An antiemetic patch, comprising:
an antiemetic composition including an antiemetic agent ranged from 1% to 15% by weight of the antiemetic composition.

13. The antiemetic patch as claimed in claim 12, wherein the antiemetic composition further includes a permeation enhancer ranged from 1% to 10% by weight of the antiemetic composition.

14. The antiemetic patch as claimed in claim 12 having a matrix type.

15. The antiemetic patch as claimed in claim 12, further comprising:
a backing layer;
a release liner; and
a matrix layer disposed between the backing layer and the release liner and including the antiemetic composition.

16. A method of treating an emesis of an individual, comprising:
applying the antiemetic patch of claim 12 to the individual, wherein the antiemetic agent has a plasma concentration sustainable in a therapeutically effective range for at least 5 days after the antiemetic patch is applied to the individual.

17. The method of claim 16, wherein the emesis is one selected from a group consisting of an acute, a delayed, an anticipatory emesis and a combination thereof.

18. The method of claim 16, wherein the individual has a skin and a mucosa, and the antiemetic patch is applied to at least one of the skin and the mucosa.

19. The method of claim 16, wherein the plasma concentration of the antiemetic agent in the therapeutically effective range has a value larger than 0.48 ng/ml/cm².

20. The method of claim 16, wherein the antiemetic patch has an area in a range of 10 cm² to 100 cm², and the treated individual has a peak plasma concentration of the antiemetic agent being larger than 6 ng/ml.
